(19) United States
(12) Patent Application Publication
(10) Pub. No.: US 2021/0316026 A1
(43) Pub. Date: Oct. 14, 2021

(54) VEHICULAR DOOR HANDLE ASSEMBLY WITH STERILIZING FEATURE

(71) Applicant: Magna Mirrors of America, Inc., Holland, MI (US)

(72) Inventor: James A. Ruse, Allegan, MI (US)

(21) Appl. No.: 17/301,772

(22) Filed: Apr. 14, 2021

Related U.S. Application Data
(60) Provisional application No. 63/009,549, filed on Apr. 14, 2020.

(51) Int. Cl. A61L 2/10; B62D 1/04; E05B 85/10; E05B 1/00

(52) U.S. Cl. CPC A61L 2/10; B62D 1/04; A61L 2202/14; E05B 1/0069; A61L 2202/11; E05B 85/10

(57) ABSTRACT

A door handle assembly for a vehicular door of a vehicle includes a base portion disposed at the vehicular door, a handle portion, and an ultraviolet (UV) radiation emitter. The handle portion includes a grasping portion configured for a user to grasp when operating the door handle assembly. The handle portion is movable relative to the base portion by a user grasping the grasping portion to actuate a latch mechanism for opening the vehicular door. The UV radiation emitter, when electrically powered, irradiates at least the grasping portion of the handle portion with UV radiation.

### CROSS REFERENCE TO RELATED APPLICATION

The present application claims the filing benefits of U.S. provisional application Ser. No. 63/009,549, filed Apr. 14, 2020, which is hereby incorporated herein by reference in its entirety.

### FIELD OF THE INVENTION

The present application relates to door handles for vehicles and, more particularly, an exterior door handle for opening a side door of a vehicle responsive to a passive entry device or system.

### BACKGROUND OF THE INVENTION

A door handle for a vehicle door typically includes a handle portion that is pivotable relative to a base portion, whereby pivotal movement of the handle portion pulls at a cable or rod to electrically trigger or move a latch mechanism to release the latch and open the door.

### SUMMARY OF THE INVENTION

A door handle assembly is configured to mount at a handle region of a vehicle door. The door handle assembly comprises a handle portion that pivotally or movably attaches at a bracket or base portion of the door and that is configured to operate a latch mechanism when a grasping portion is grasped or pulled or moved by a user. The door handle assembly comprises an ultraviolet (UV) radiation or light source that, when operated irradiates at least the grasping portion with UV radiation or light to sterilize at least the grasping portion. The UV radiation source may be at the bracket or handle pocket region of the door (so that the UV radiation source, when activated, directs UV radiation toward the handle portion) or the UV radiation source may be at one or both ends of the handle portion (so that the UV radiation source, when activated, directs UV radiation along the handle portion, with the handle portion having an outer shell that is transmissive to the UV radiation).

Optionally, a UV radiation source or a plurality of UV radiation sources may be disposed at an interior door handle of the vehicle to irradiate the interior handle portion with UV radiation or light to sanitize the interior vehicle door handle. Optionally, a UV radiation source or a plurality of UV radiation sources may be disposed at the steering wheel of the vehicle to irradiate the steering wheel with UV radiation or light to sanitize the steering wheel. Optionally, a UV radiation source or a plurality of UV radiation sources may be disposed at the center console of the vehicle to irradiate the touch screen or controls and/or the gear selector with UV radiation or light to sanitize other components that are often touched by the driver or passenger of the vehicle.

The system or apparatus or assembly provides for sanitizing and disinfecting often touched surfaces of a vehicle, such as door handles, steering wheel, controls, etc., via UV light or radiation emitters, such as light emitting diodes (LEDs) and light guides, which provide UV radiation at the surface from within the component or via radiation emitted by a UV LED spaced from the component. The system(s) provide for safer use of the vehicle, and may be particularly suited for vehicles where multiple people interface with the vehicle, such as fleet vehicles, taxis, valet parked vehicles, test drivers, rental vehicles, passenger van shuttles, crew vehicles and the like.

These and other objects, advantages, purposes and features of the present invention will become apparent upon review of the following specification in conjunction with the drawings.

### BRIEF DESCRIPTION OF THE DRAWINGS

FIG. 1 is a perspective view of a vehicle with a door handle assembly;

FIG. 2 is a perspective view of a door handle assembly having a UV light source;

FIG. 3 is a top plan view of the door handle assembly of FIG. 2;

FIG. 4 is a sectional view of the door handle assembly of FIG. 2;

FIGS. 5-7 are exploded views of the door handle assembly of FIG. 2;

FIG. 8 is another sectional view of the door handle assembly, shown with an optional core reinforcement;

FIG. 9 is a plan view of a steering wheel having a UV light source;

FIG. 10 is a perspective view of the steering wheel of FIG. 9;

FIGS. 11-13 are exploded views of the steering wheel of FIG. 9;

FIG. 14 is a schematic showing the light guide for guiding UV light along the steering wheel;

FIG. 15 is an enlarged perspective view of a portion of the steering wheel;

FIGS. 16 and 17 are perspective views of another steering wheel having a UV light source;

FIGS. 18 and 19 are exploded views of the steering wheel of FIGS. 16 and 17;

FIGS. 20 and 21 are perspective views of another steering wheel having a UV light source at controls at the steering wheel;

FIG. 22 is an enlarged perspective view of the controls and the UV light source directed at the controls of the steering wheel of FIGS. 20 and 21; and

FIG. 23 is another perspective view of the steering wheel of FIGS. 20 and 21.

### DESCRIPTION OF THE PREFERRED EMBODIMENTS

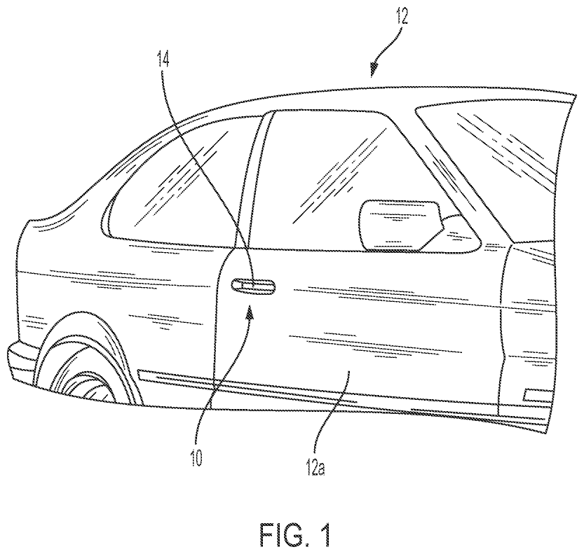

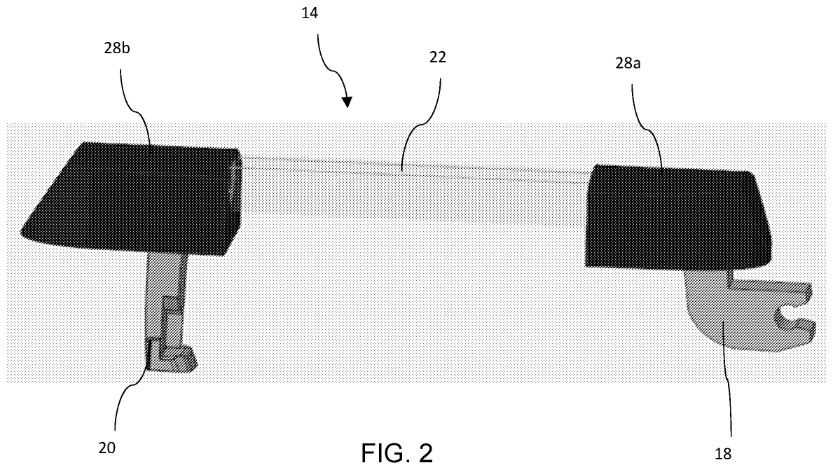

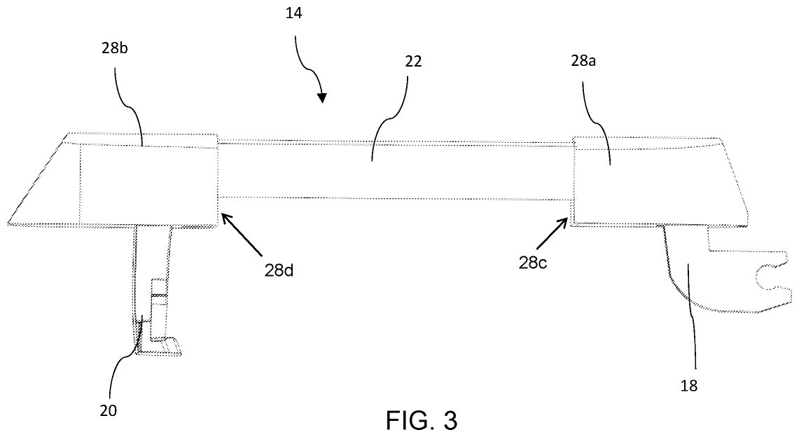

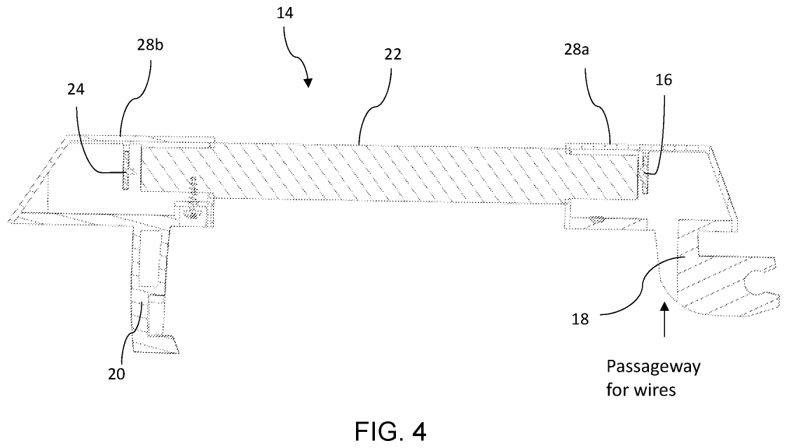

Referring now to the drawings and the illustrative embodiments depicted therein, a vehicle door handle assembly 10 is mountable to a door 12a of a vehicle 12 and operable to release a latch mechanism of the door 12a to open the vehicle door (FIG. 1). The vehicle door handle assembly 10 includes a handle portion 14 that is disposed at the door 12a and that is fixedly mounted at a base such as the door or to a bracket mounted to the door. The handle portion is movably mounted at the handle region of the vehicle door (such as pivotally mounted), whereby movement of the handle portion by a user opens the vehicle door. Optionally, the handle assembly includes a touch sensor and when the handle portion is contacted (such as by a user holding a key fob associated with the vehicle), the touch sensor sends a signal to release the latch mechanism. The door handle assembly includes an ultraviolet (UV) light or radiation emitter, such as a light-emitting light emitting diode (LED) 16, that emits UV light or UV radiation at the handle portion to sanitize the handle portion.

In the illustrated embodiment, the handle portion 14 comprises a hinge portion 18 at one end and a tower portion 20 (or actuating portion) at the other end (that is connected to a cable or latching mechanism such that movement of the actuating portion actuates the mechanism to open the vehicle door), with a grasping portion 22 disposed therebetween for a user to grab and operate the door handle. The grasping portion 22 comprises a light guide and a UV light transmitting outer material. The UV LED 16 is disposed at one end of the grasping portion so that light emitted by the UV LED is transmitted along the grasping portion via the light guide and dispersed to and through the UV light transmitting outer material of the grasping portion. The UV LED emits UV light having a wavelength in the UV spectral band of radiation, such as between 240 nm and 310 nm, or such as between 255 nm and 290 nm. Such a UV LED may provide for 90 percent elimination of bacteria and viruses when operating at 2 to 8 mWs/cm². For example, the UV LED may provide an effective output of nearly 10 mW·s/cm² when operating at 10 mW for several seconds, such as up to about 60 seconds (assuming a 60 percent effectiveness and 40 cm² grasping portion surface). The UV LED may comprise an LED of the types provided by Seoul Viosys Co. Ltd.

A UV detector 24 is disposed at the other end to detect the UV light emitted by the UV LED when the UV LED is actuated or powered. The detector operates to determine the degree of UV light emitted by the UV LED to evaluate the effectiveness of the UV LED and to determine when the UV LED should be replaced due to aging. For example, the UV detector may be in communication with an electronic control unit (ECU) of the vehicle and communicate a signal to the ECU indicating the need to replace the UV LED, such as when the effectiveness of the UV LED is determined to be below a threshold value or if the UV LED has been activated a threshold number of times.

The grasping portion may comprise a UV light transmitting material, such as a polymethyl methacrylate (PMMA) or acrylic material or polycarbonate material that is transmissive of UV light or UV radiation emitted by the UV LED. Such a material may provide transmittance of greater than 50 percent, such as greater than 75 percent, such as up to about 90 percent, of UV light incident thereat. The material is selected to provide the desired or appropriate degree of reflection and refraction so that the transmitted light is dispersed within the grasping portion and diffusely transmitted through the outer surface for substantially uniform disinfecting and sanitizing of the outer surface of the grasping portion. Optionally, the material may comprise liquid crystal/polymer microsphere composites or microbeads or facets or the like that provide diffuse and uniform dispersion of the UV radiation at the outer surface of the grasping portion. The grasping portion may include a light guide or light guiding structure within the grasping portion to guide the UV light outward toward and through the exterior part of the grasping portion to irradiate and sanitize the outer surface of the grasping portion.

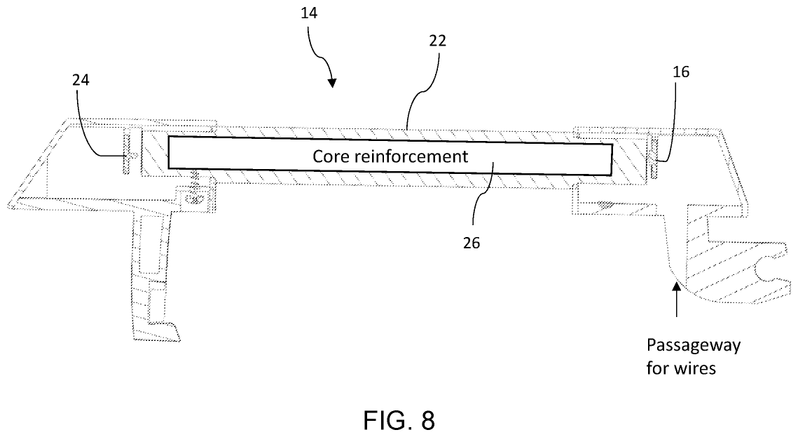

Thus, the door handle assembly comprises an emitter at one end of the grasping portion, and optionally a detector at the other end to evaluate the strength or effectiveness of the UV light emitted and thus determine the replacement timeframe due to UV aging. The grasping portion comprises a translucent or UV light transmitting element. Optionally, and such as shown in FIG. 8, the grasping portion may comprise a core reinforcement element 26 (comprising an engineering plastic thermoplastic or the like). The core reinforcement element 26 may be colored to match the cover elements 28a, 28b at the ends of the handle portion so the grasping portion does not appear translucent or transparent. The UV light transmitting material of the grasping portion may be overmolded over and around the core reinforcement element or may be part of an outer cover portion of the grasping element that may be attached at the core element, such as via adhesive or fasteners. The UV radiation may pass along and within the core element or may pass along and within the cover portion or outer portion of the grasping portion outboard of the core element. The grasping portion or cover portion may comprise a replaceable translucent optical piece.

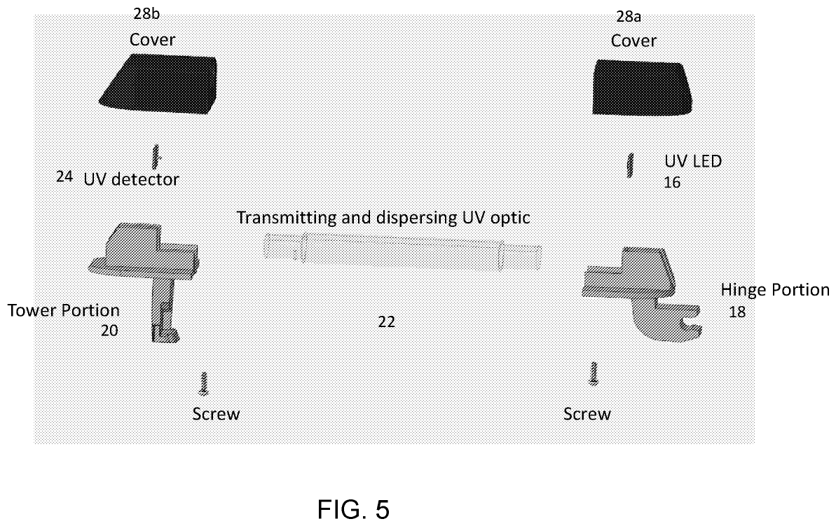

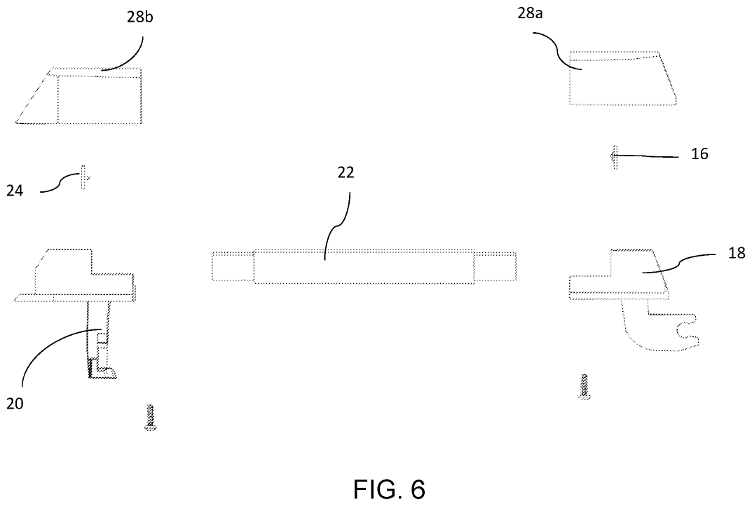

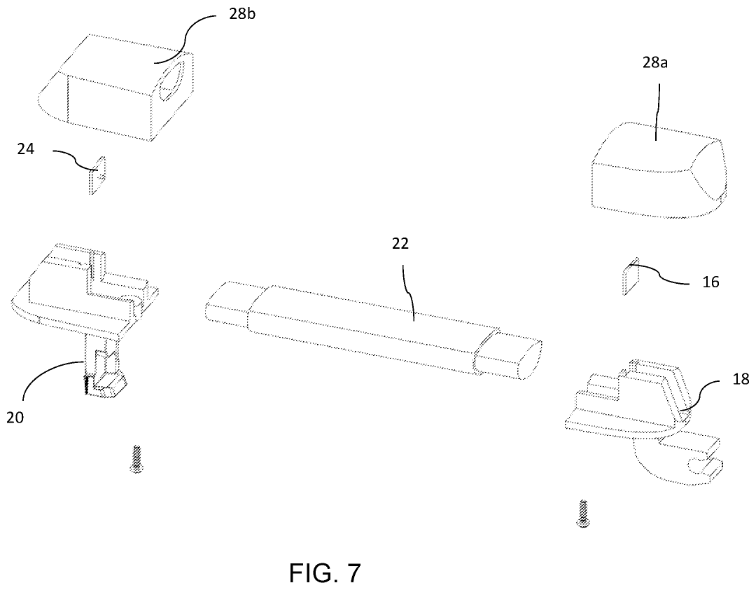

Furthermore, UV light transmitting material may be disposed across the entirety of the outer surface of the grasping portion (FIG. 5) or the UV light transmitting material may be selectively disposed or exposed at the grasping portion such as to direct incident light at a non-translucent portion of the grasping portion of the door handle. For example, the outer surface of the grasping portion may substantially comprise a non-translucent material (such as to match the body color of the vehicle) and the UV light transmitting material may be disposed as a core of the grasping element with an exposed portion at the outer surface of the grasping portion that enables UV light to travel from the UV LED through the UV light transmitting material and outward from the exposed portion to direct incident light on the non-translucent portion of the grasping portion. For example, the exposed portion may comprise a raised ridge across the surface of the grasping portion of UV light transmitting material that directs UV light over the surface of the handle.

Optionally, the door handle assembly may comprise a UV LED that emits light (when powered) directly onto the exterior surface of the grasping portion (such that the grasping portion may comprise any suitable material). For example, the UV LED or LEDs may be disposed at the base or pocket region at the door panel and directed outward toward the grasping portion and/or the UV LED or LEDs may be disposed at one of the cover elements 28a, 28b and directed along the exterior surface of the grasping portion. For example, the UV LED or LEDs may be disposed at or behind a surface 28c, 28d of the respective cover elements 28a, 28b to direct UV light at the grasping portion 22. Similar to the embodiment discussed above, one surface may house a UV LED and one surface may house a UV detector for determining the strength of the UV light emitted by the LED and determining whether the UV LED should be replaced due to LED aging.

The UV LED or LEDs may be episodically operated to disinfect or sanitize the door handle, such as after a user has touched the door handle or opened or closed the vehicle door. For example, the system may determine when a user has opened the door, and may operate the UV LED or LEDs for a period of time after the user has opened the door, and may delay actuation of the UV LED or LEDs to allow the user time to get into the vehicle so that the user is not present at the exterior door handle during operation of the UV LED or LEDs. Additionally, the system may activate when the user operates an unlock or lock function via a key fob of the vehicle. For example, a user may activate the unlock function and the system may emit UV light at or onto the door handles to sanitize the door handle prior to the user grabbing the handle to open the door. If the vehicle unlocks responsive to sensing an authorized key fob within range of the vehicle, the system may first emit UV light at or onto the door handles prior to or in conjunction with unlocking the door responsive to sensing the authorized key fob within range of the vehicle or within a threshold distance from the vehicle.

Thus, the vehicular door handle has automatic Ultraviolet LED sanitization, which may use pocket lighting and/or an at least partially translucent handle with UV LEDs embedded in the handle for directing UV light along and within the UV light transmitting material of the handle. For direct UV irradiation (e.g., from a UV LED at the pocket region), the UV LED is disposed behind a cover or lens to seal the UV LED at the vehicle door. The cover or lens comprises a translucent or transparent cover or lens and functions to provide a desired illumination pattern that provides uniform or substantially uniform irradiation of the handle portion and that avoids hotspots (areas of higher intensity UV radiation) at the handle portion. The cover or lens may utilize aspects of the lighting modules or devices described in U.S. Pat. Nos. 9,761,144; 9,487,143 and/or 8,801,245, and/or U.S. Publication Nos. US-2019-0106051 and/or US-2015-0224919, which are hereby incorporated herein by reference in their entireties.

Also, the door or pocket region or UV device or cover or lens may include baffles or structure that limits or precludes light emitted from the UV LED from being incident at the eyes of a person at the side of the vehicle and opening the vehicle door (or at the rear of the vehicle and opening a rear lift gate or tailgate or the like). For example, the pocket region may have a structure disposed over the UV LED so that light emitted by the UV LED is absorbed or blocked or reflected downward by the structure and cannot be emitted upward toward where the eyes of a person would be when that person is opening the door. The system may avoid directing UV light at the head or eye level of persons near the vehicle in other ways, such as determining when the rear liftgate is in an opened upward position (and thus a UV LED that is normally pointed downward to direct light at a handle of the rear liftgate is now pointed horizontally outward from the vehicle) and preventing the UV LED from being operated.

The door handle assembly may utilize aspects of the assemblies described in U.S. Publication No. US-2019-0106051, which is hereby incorporated herein by reference in its entirety. The door handle assembly may comprise any suitable type of door handle assembly, and may include or incorporate aspects of the door handle assemblies and lighting devices described in U.S. Pat. Nos. 6,550,103; 7,407,203; 8,333,492; 8,786,401 and/or 8,801,245, and/or U.S. Publication Nos. US-2020-0130646; US-2010-0088855 and/or US-2010-0007463, which are all hereby incorporated herein by reference in their entireties.

The door handle assembly is operable to open the vehicle door when a user grasps the door handle portion at the side of the vehicle door. The door handle assembly may also be operable in conjunction with a passive keyless entry or other sensing system that is operable to determine whether or not the person at the vehicle door is authorized for entry into the vehicle, and may only open the vehicle door when that system recognizes the user or key fob or transmitting device associated with the owner or authorized user of the vehicle. Optionally, the door handle assembly may be associated with or in communication with a door zone module, such as by utilizing aspects of the vehicle door systems described in U.S. Publication No. US-2010-0007463, which is hereby incorporated herein by reference in its entirety.

Although shown and described above as being part of a vehicular exterior door handle assembly, aspects of the UV light system are suitable for use at other components of a vehicle that are often contacted by the user or driver or passenger of the vehicle. For example, one or more UV LEDs may be disposed at an interior door handle assembly of a vehicle and/or at the dashboard or center console controls of the vehicle (such as at a touch screen or control buttons and/or knobs or at the gear selector or at the seat belt or at the turn signal actuator). The UV LEDs are placed in close proximity to the item to be irradiated so that the UV LEDs can effectively sanitize and disinfect the items in a short period of time and without the UV radiation being incident on the eyes of a person in the vehicle. For example, a UV LED directed at the interior door handle may be embedded in a door handle pocket behind a translucent lens that directs uniform light at and across the surface of the interior door handle. A UV LED directed at other touch points within the vehicle may similarly be situated at a surface of the vehicle near the element to be sanitized or a UV LED may be positioned to disinfect larger areas or multiple components of the vehicle (such as at an overhead console in the vehicle cabin to direct light at a substantial portion of the dashboard and/or driver area of the vehicle).

Optionally, the UV LEDs may be similarly situated to the door handle assembly discussed above, where the UV LEDs are contained within an element of the component to be sanitized and the component is at least partially translucent (formed from UV light transmitting material) so that with the UV LED activated, the UV light transmitting material may guide UV light through the component to the surface to be sanitized. For example, the gear selector may be at least partially translucent with a UV LED disposed within for directing UV light along and within UV transmitting material of the gear selector. Control buttons, such as at the center console, may be at least partially translucent and backlit with UV LEDs.

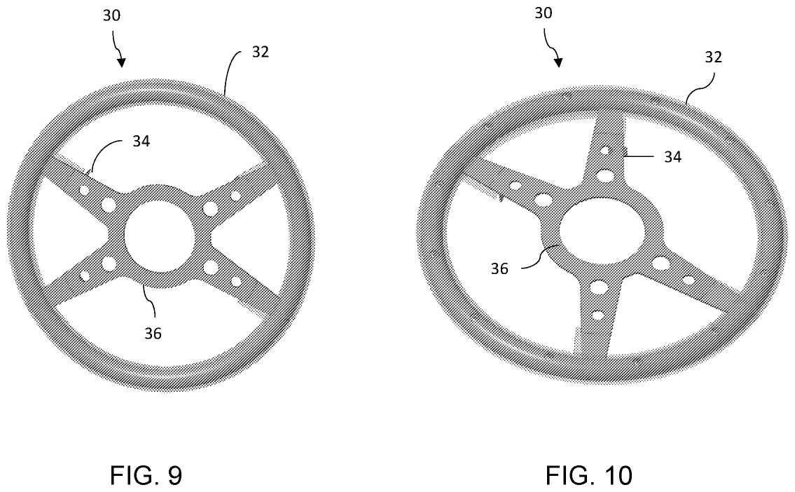

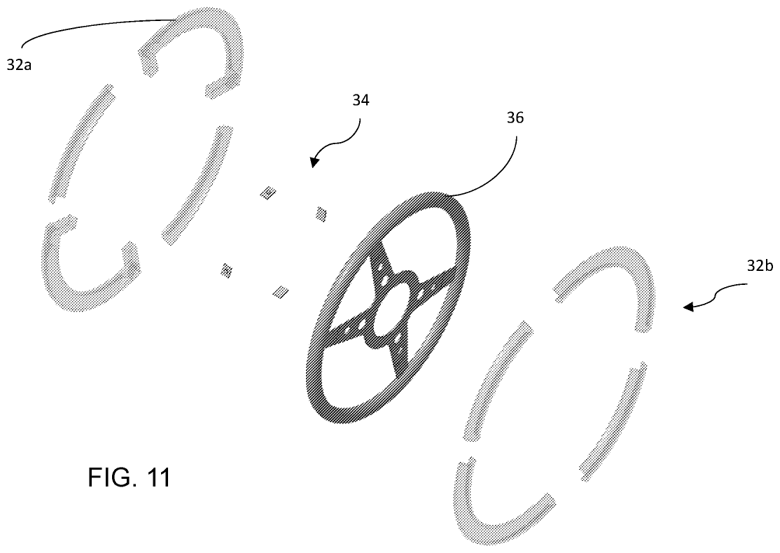

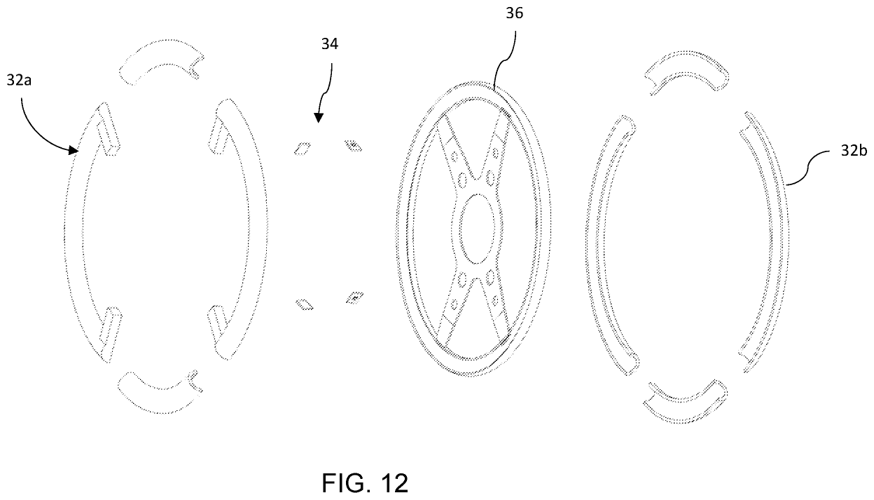

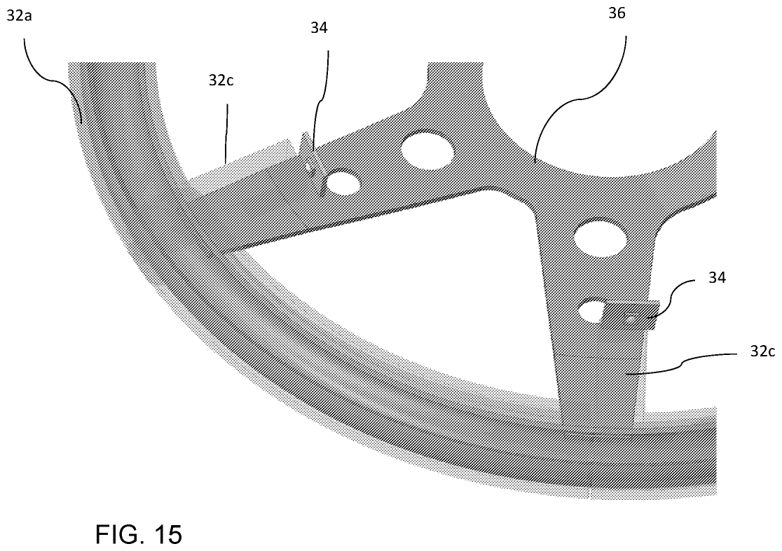

Optionally, and such as shown in FIGS. 9-15, a UV lighting device may be disposed at a steering wheel 30 of the vehicle to sanitize or disinfect the grasping portion 32 of the steering wheel. In the illustrated embodiment, the UV lighting device comprises a plurality of individual UV LEDs 34 disposed radially inboard from the circular grasping portion 32, such as at respective spokes of the steering wheel structure 36 (comprising a central hub and spokes that support the circular structure of the grasping portion). The steering wheel grasping portion 32 comprises a translucent or UV light transmissive cover and light guide (having an inner portion 32a and an outer portion 32b) that encases the grasping portion of the steering wheel and provides an outer cover or surface of the grasping portion. The LEDs are disposed at radially inboard extending passageways 32c of the cover 32, whereby UV light emitted by the UV LEDs 34 passes along the passageways 32c and is guided or reflected by angled reflective elements or mirrors 32d to direct or reflect the UV light along the partial circular cover portions where it passes through the UV light transmitting material to sanitize and disinfect the outer surface of the steering wheel.

One or both passageways 32c (of each section) may house the UV LEDs so that UV light is transmitted from both ends of each section of the grasping portion 32. Additionally, one or both passageways 32c (of each section) may house a UV light detector (in a similar manner to the door handle assembly described above) to detect the degree or intensity of the UV light transmitted within the grasping portion and to determine when the UV LED should be replaced. Thus, the grasping portion 32 substantially conforms to the steering wheel or maintains a cover surface at the grasping portion of the steering wheel substantially flush against the grasping surface of the steering wheel so as not to significantly increase the grip circumference of the steering wheel. Optionally, the UV LEDs may be disposed within the partial circular cover portions of the grasping portion to emit light directly within the UV light transmitting material without use of mirrors, such as at a raised portion of the cover accommodating the UV LED.

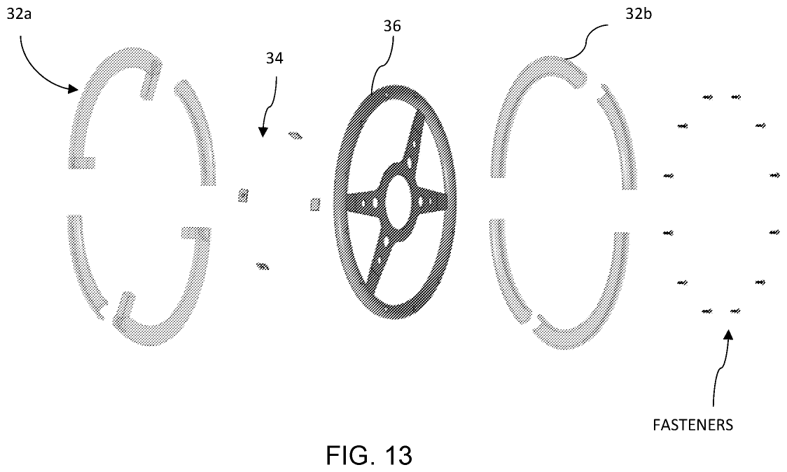

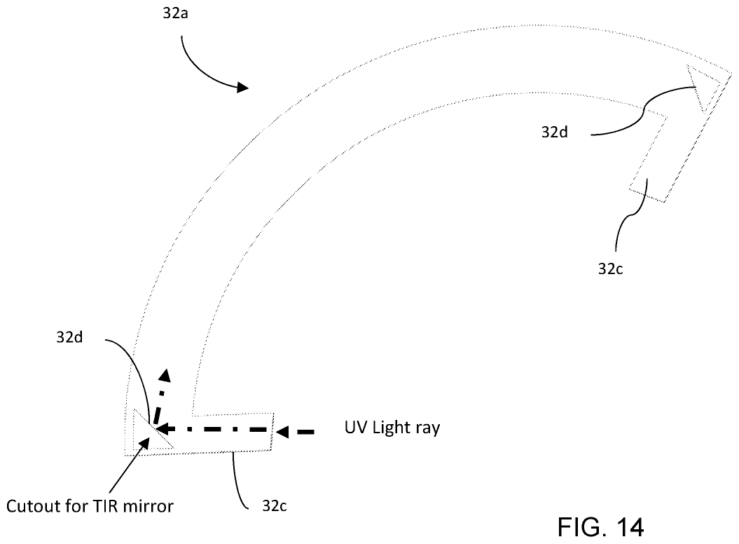

As shown in FIGS. 10 and 13, the cover portions 32a, 32b may be fastened together at the steering wheel inner structure 36 to secure the outer cover around the wheel portion, with the UV LEDs disposed at respective spokes of the steering wheel structure. Although shown as retained via fastening screws, the inner and outer portions may otherwise be retained at the steering wheel structure, such as via snap attachment or a translucent adhesive. As shown in FIG. 14, sections of the steering wheel may have a UV LED and mirror at each end such that UV light is directed along the wheel portion from both ends. Optionally, each section or segment of the wheel may have its own respective LED and mirror with the corner of the generally L-shaped light path being turned using TIR (total internal reflection).

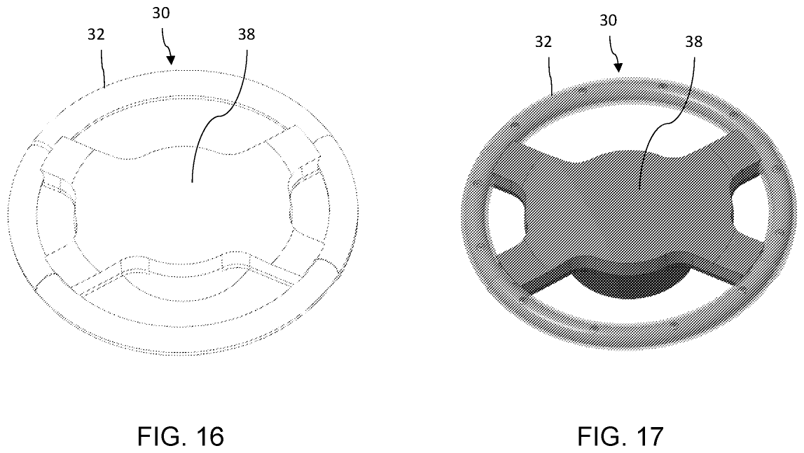

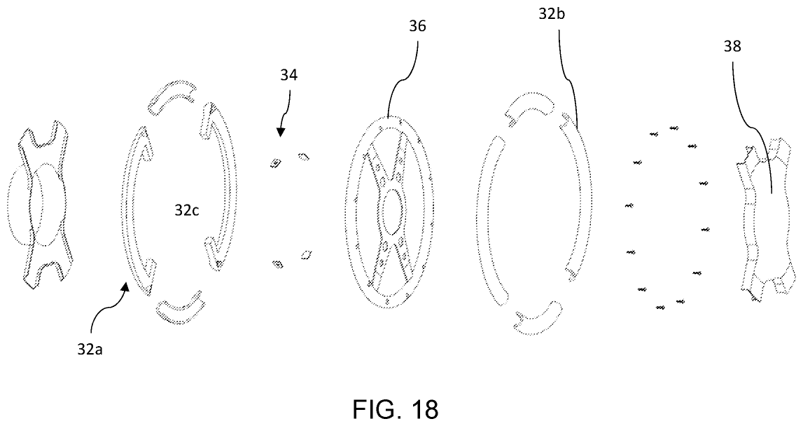

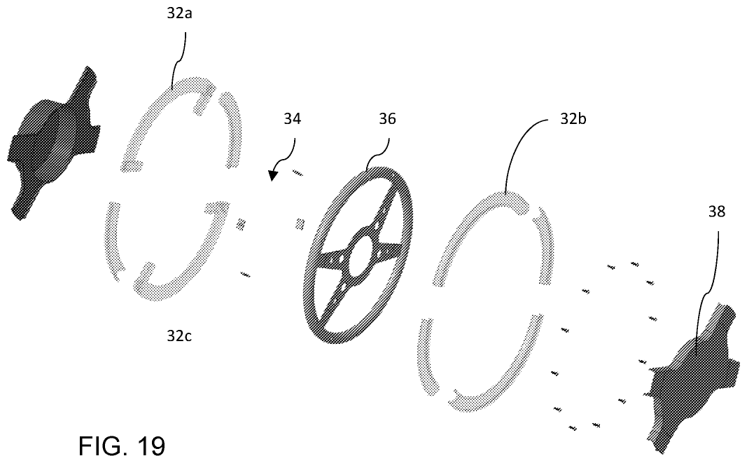

Optionally, and such as shown in FIGS. 16-19, the steering wheel 30 may comprise an airbag or other structure or electronic component(s) at the center region, whereby a central cover element 38 is disposed at the outer (driver facing) portion of the steering wheel to cover the center hub of the structure 36 and the UV LEDs 34 and passageways 32c from view by the driver of the vehicle.

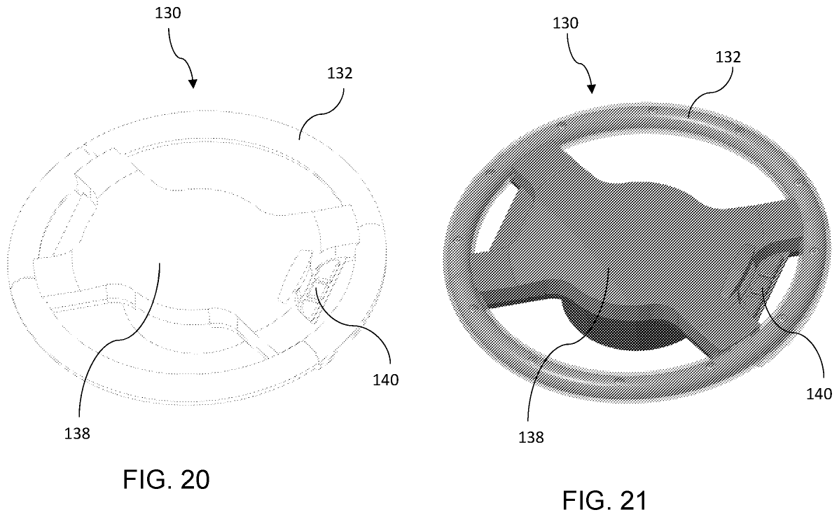

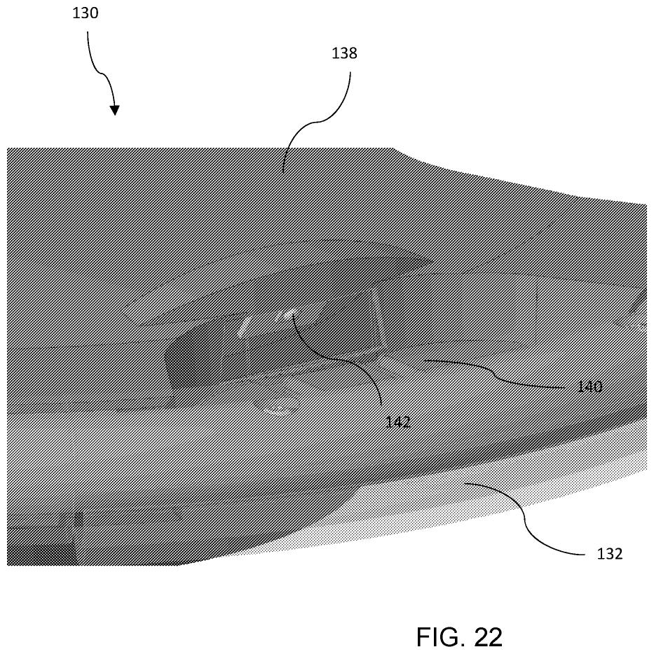

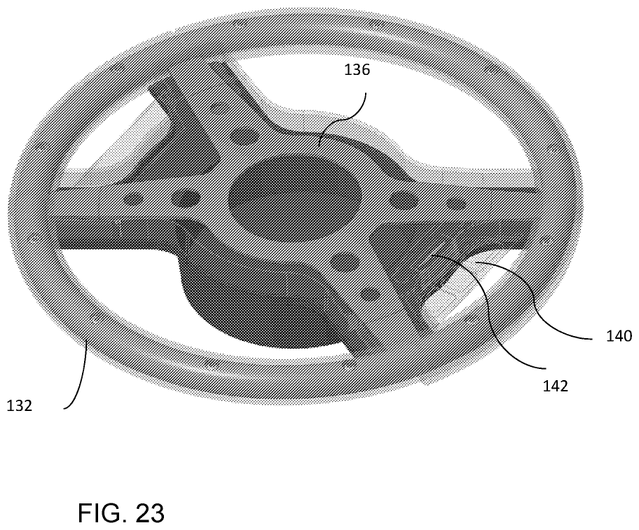

Optionally, and such as shown in FIGS. 20-23, the steering wheel 130 includes steering wheel mounted controls (such as for cruise control, radio, telephone, etc.) 140, and one or more UV LEDs 142 may be disposed at the central hub region and directed through an aperture of the steering wheel cover (see FIG. 22) so as to emit light directly onto the user inputs or control buttons or toggles or switches at the steering wheel. The steering wheel may include a UV radiation transmissive cover or element so that, when the UV LED 142 is electrically powered, substantially uniform or diffuse UV radiation is provided over the controls, such as in a similar manner as the door handle cover or lens, discussed above, such that UV radiation emitted by the UV LED 142 is absorbed or blocked or reflected toward the controls so that it is not directed toward the driver of the vehicle. The steering wheel 130 also includes UV LEDs 134 that emit light that is guided along the wheel cover portion 132 (disposed about the wheel structure 136) to disinfect or sterilize the circular wheel portion, such as in a similar manner as described above.

The UV LEDs of the steering wheel (as well as UV LEDs that are directed toward the center console controls or the like) may be operated or powered after the vehicle is used or responsive to a user input. For example, activation of the UV LEDs may be responsive to parking the vehicle and the driver exiting the vehicle (which may be determined by a driver monitoring system or other suitable means for determining when the driver has exited the vehicle) or responsive to a user input such as a lock function at a key fob of the vehicle, so that the UV light is emitted when the vehicle is not being driven and before the next use of the vehicle. The system may limit or preclude operation of the UV LEDs at the interior components when a driver or passenger is determined to be present in the cabin of the vehicle.

A UV light emitting device may be disposed at a steering wheel or other frequently touched component of a vehicle in a variety of forms. For example, the steering wheel cover discussed above may be permanently disposed or embedded in the steering wheel of the vehicle or the steering wheel cover may be removably attached to the steering wheel of the vehicle such as via snap attaching the inner portion to the outer portion around the grasping portion of the steering wheel to sanitize the steering wheel before use (and removing the cover before operating the vehicle).

Thus, devices and methods include UV light emitters (such as UV light-emitting LEDs or vertical-cavity surface-emitting lasers (VCSEL) or other suitable UV light emitters) at and/or near exterior door handles of a vehicle, the steering wheel of a vehicle, and other touchpoints of a vehicle, such as the gear selector, control buttons, seat belt, steering wheel stalk controls, etc., for sanitizing the surfaces of vehicular elements using UV light.

These systems are desirable such as for fleet vehicles (e.g., taxis, rental cars, delivery trucks) or other vehicles that may frequently be used by different drivers and passengers.

Changes and modifications to the specifically described embodiments may be carried out without departing from the principles of the present invention, which is intended to be limited only by the scope of the appended claims as interpreted according to the principles of patent law.

1. A door handle assembly for a vehicular door, said door handle assembly comprising:
a base portion disposed at a vehicular door;
a handle portion having a grasping portion configured for a user to grasp when operating the door handle assembly, wherein the handle portion is movable relative to the base portion by a user grasping the grasping portion to actuate a latch mechanism for opening the vehicular door; and
an ultraviolet (UV) radiation emitter that, when electrically powered, irradiates at least the grasping portion of the handle portion with UV radiation.

2. The door handle assembly of claim 1, wherein the UV radiation emitter is disposed at the handle portion and emits UV radiation along the grasping portion.

3. The door handle assembly of claim 2, wherein the grasping portion comprises a UV radiation transmissive material, and wherein the UV radiation emitter emits UV radiation at an end of the grasping portion so that the emitted UV radiation passes along the grasping portion and outward through an outer surface of the grasping portion.

4. The door handle assembly of claim 3, wherein the UV radiation transmissive material is disposed around a reinforcing core element of the grasping portion.

5. The door handle assembly of claim 3, wherein an outer part of the grasping portion does not comprise the UV radiation transmissive material to limit transmission of UV radiation outward toward a person at the vehicular door.

6. The door handle assembly of claim 1, wherein the UV radiation emitter is disposed at the base portion and emits UV radiation toward the grasping portion.

7. The door handle assembly of claim 6, wherein a UV radiation transmissive cover is disposed at the base portion and directs the emitted UV radiation toward the grasping portion.

8. The door handle assembly of claim 7, wherein the UV radiation transmissive cover diffuses UV radiation emitted by the UV radiation emitter.

9. The door handle assembly of claim 7, wherein a non-UV radiation transmissive element is disposed at the base portion, the non-UV radiation transmissive element limiting emitted UV radiation from being emitted upward toward a head region of a person at the vehicular door.

10. The door handle assembly of claim 1, wherein the door handle assembly comprises an exterior door handle assembly disposed at an exterior portion of the vehicular door.

11. The door handle assembly of claim 1, wherein the door handle assembly comprises an interior door handle assembly disposed at an interior portion of the vehicular door.

12. The door handle assembly of claim 1, wherein the UV radiation emitter is electrically powered responsive to a user input at a key fob of the vehicle.

13. The door handle assembly of claim 1, wherein the UV radiation emitter is electrically powered responsive to the vehicular door being moved from a closed position to an opened position.

14. The door handle assembly of claim 13, wherein the UV radiation emitter is electrically powered a threshold amount of time after the vehicular door is moved from the closed position to the opened position.

15. A steering wheel assembly for a vehicle, said steering wheel assembly comprising:
a steering wheel, the steering wheel comprising a grasping portion; and
an ultraviolet (UV) radiation emitter that, when electrically powered, irradiates the grasping portion of the steering wheel with UV radiation.

16. The steering wheel assembly of claim 15, comprising a cover portion that is disposed at the grasping portion and comprises a UV radiation transmissive material, wherein the UV radiation emitter emits UV radiation at an entry of the cover portion so that the emitted UV radiation passes along the cover portion and outward through an outer surface of the cover portion.

17. The steering wheel assembly of claim 16, wherein the cover portion includes a reflector that directs UV radiation from the entry in a direction along the grasping portion.

18. The steering wheel assembly of claim 17, wherein the cover portion includes (i) an inner portion that includes the entry of the cover portion and (ii) an outer portion that snap attaches to the inner portion to retain the cover portion at the steering wheel.

19. The steering wheel assembly of claim 16, wherein an outer part of the cover portion comprises a material that is not transmissive of UV radiation to limit emitted UV radiation from being emitted toward a person at the steering wheel assembly.

20. The steering wheel assembly of claim 15, further comprising steering wheel-mounted controls and another UV radiation emitter that, when electrically powered, irradiates the steering wheel-mounted controls with UV radiation.

21. The steering wheel assembly of claim 15, wherein the UV radiation emitter is electrically powered responsive to a determination that no person is present at an interior portion of the vehicle.

22. A door handle assembly for a vehicular door, said door handle assembly comprising:
a base portion disposed at a vehicular door;
a handle portion having a grasping portion configured for a user to grasp when operating the door handle assembly, wherein the handle portion is movable relative to the base portion by a user grasping the grasping portion to actuate a latch mechanism for opening the vehicular door; and
wherein the grasping portion comprises a UV radiation transmissive material, and wherein the UV radiation transmissive material is disposed around a reinforcing core element of the grasping portion;
an ultraviolet (UV) radiation emitter disposed at the handle portion, wherein the UV radiation emitter, when electrically powered, emits UV radiation at an end of the grasping portion so that the emitted UV radiation passes along the grasping portion and outward through an outer surface of the grasping portion to irradiate at least the grasping portion of the handle portion with UV radiation; and
wherein an outer part of the grasping portion does not comprise the UV radiation transmissive material to limit transmission of UV radiation outward toward a person at the vehicular door.

23. The door handle assembly of claim 22, wherein the door handle assembly comprises an exterior door handle assembly disposed at an exterior portion of the vehicular door.

24. The door handle assembly of claim 22, wherein the door handle assembly comprises an interior door handle assembly disposed at an interior portion of the vehicular door.

25. The door handle assembly of claim 22, wherein the UV radiation emitter is electrically powered responsive to a user input at a key fob of the vehicle.

26. The door handle assembly of claim 22, wherein the UV radiation emitter is electrically powered responsive to the vehicular door being moved from a closed position to an opened position.

27. The door handle assembly of claim 26, wherein the UV radiation emitter is electrically powered a threshold amount of time after the vehicular door is moved from the closed position to the opened position.
